(19) United States
(12) Patent Application Publication
(10) Pub. No.: US 2026/0130630 A1
(43) Pub. Date: May 14, 2026

(54) METHOD FOR POSITIONING A PATIENT COUCH, PATIENT COUCH AND POSITIONING ARRANGEMENT

(71) Applicant: Siemens Healthineers AG, Forchheim (DE)

(72) Inventors: Andreas DEINLEIN, Bayreuth (DE); Robert JACKO, Uzovce (SK)

(73) Assignee: Siemens Healthineers AG, Forchheim (DE)

(21) Appl. No.: 19/384,285

(22) Filed: Nov. 10, 2025

(30) Foreign Application Priority Data
Nov. 11, 2024 (DE) 10 2024 210 804.9

(51) Int. Cl. A61B 5/00; A61B 5/055; A61B 6/03; A61B 6/04; A61N 5/10

(52) U.S. Cl. CPC A61B 5/706; A61B 5/055; A61B 5/704; A61B 6/032; A61B 6/0407; A61B 6/0487; A61N 5/1078

(57) ABSTRACT

A method for positioning a mobile patient couch relative to a stationary medical device, comprises: capturing, by an optical sensor, a first optical marker in an image; providing the image to a controller; determining, by the controller, a position and/or orientation of the mobile patient couch relative to the stationary medical device based on the image of the first optical marker and/or of the first optical marker comprised by the image; and positioning the mobile patient couch by controlling the drive unit and/or the steering unit based on the position and/or orientation of the mobile patient couch relative to the stationary medical device.

### CROSS-REFERENCE TO RELATED APPLICATION(S)

The present application claims priority under 35 U.S.C. § 119 to German Patent Application No. 10 2024 210 804.9, filed Nov. 11, 2024, the entire contents of which is incorporated herein by reference.

### FIELD

One or more example embodiments of the present invention relate to a method for positioning a patient couch relative to a stationary medical facility (also referred to as a stationary medical device), wherein the patient couch comprises a control facility (also referred to as a control device), a drive and/or a steering unit, wherein the drive and/or the steering unit is able to be controlled by the control facility on the basis of control signals. One or more example embodiments of the present invention further relate to a patient couch for application of the method and to a positioning arrangement with the patient couch.

### BACKGROUND

Mobile patient couches are known per se. On the one hand these can interact with a medical imaging system during an image data acquisition or also be moved detached from the imaging system.

In order to facilitate pushing or driving of the patient couch, the chassis of the mobile patient couch can for example comprise four freely pivotable casters, each on a corner of the patient couch, and a fifth wheel aligned straight and centrally in the middle between the casters. This fifth wheel is pressed via a spring mechanism against the floor and functions as a guide wheel. The fifth wheel makes possible in a manual operating mode of the patient couch to simply push the couch in a straight line or around tight curves and, while stationary, to turn the patient couch about the fifth wheel. As an alternative the fifth wheel can be equipped with a drive motor, for a motor-assisted or autonomous operating mode of the patient couch, which can provide motorized support for pushing the patient couch or can act on its own. It has previously been the task of the operator to predetermine the direction of the couch movement by turning the patient couch about the fifth wheel. The four casters align themselves in such cases according to the direction predetermined by the user and follow the manual instructions. Such a patient couch is known for example from the German utility model DE 202016007430.

Such mobile patient couches thus provide the opportunity to place a patient outside an examination room, i.e. detached from the medical imaging system, onto the patient couch and/or, for a magnetic resonance examination for example, to fit the coils (e.g. head, extremities or body coils) that might be needed for image data acquisition.

Subsequently the mobile patient couch must be brought to the medical imaging system. To do this, as mentioned at the outset, an operator must push the patient couch along with the patient located on it into the examination room and then align it as well as possible at a docking point of medical imaging system or the patient couch must drive itself autonomously to the docking point.

The patient couch must be driven at a speed that is as low as possible to the docking point, so that any sudden shock is avoided or suppressed as far as possible. The patient couch, for operation at the medical imaging system, frequently has a docking facility (also referred to as a dockeing device) comprising a docking tongue. So that a docking process can even begin and where necessary be able to be supported mechanically or hydraulically, the patient couch and in particular the docking tongue of the docking facility must be brought to the docking point with an alignment within an acceptance angle of −7°to +7°. Ideally during the approach an alignment within an angle of −2°to +2°should be achieved in order to minimize a lateral jerk caused by an automatic further alignment of the patient couch to 0°during the docking process.

The journey of the patient couch within a building and/or a room can also lead to discomforts for the patient. Frequently such journeys take place in restricted environments, so that they can result in collisions with objects in the environment and/or frequent forwards, backwards and/or sideways maneuvering is needed.

The discomforts for the patient during the journey, in particular during positioning in front of the scanner and/or alignment for docking, have previously had to be taken into account however.

### SUMMARY

An object of one or more example embodiments of the present invention is to provide alternate mechanisms and/or means that allow patient comfort during travel, steering and/or positioning of a mobile patient couch to be enhanced. In particular an object of one or more example embodiments of the present invention is to provide mechanisms and/or means that make possible a reliable and exact positioning of the patient couch at a medical facility.

At least this object is achieved by a method for positioning a patient couch, by a patient couch and a positioning arrangement. Preferred and/or alternative advantageous embodiment variants are the subject matter of the dependent claims, the description and the enclosed figures.

An inventive way in which at least the aforementioned object is achieved will be described below with regard to the claimed method. Features, advantages or alternative forms of embodiment mentioned here are each also able to be transferred to the other claimed subject matter and vice versa. In particular physical claims (which are directed for example to a method) can also be developed with features which are described or claimed in conjunction with one of the apparatuses. The corresponding functional features of the method are embodied in such cases by corresponding physical modules or units.

Independent of the grammatical term usage, individuals with male, female or other gender identities are included within the term.

One or more example embodiments of the present invention relate, in a first aspect, to a method for positioning a patient couch relative to a stationary medical facility (also referred to as a stationary medical device), wherein the patient couch comprises a control facility (also referred to as a control device or controller), a drive and/or a steering unit, wherein the drive and/or the steering unit is able to be controlled by the control facility on the basis of control signals, wherein the patient couch has an optical sensor facility (also referred to as an optical sensor device), which is embodied to detect images of a camera environment, wherein at least one first optical marker is arranged in a floor area in an environment of the medical facility (or medical device), characterized in that the sensor facility detects the first optical marker in an image and provides the image to the control facility, wherein the control facility determines, based on the image of the first marker provided and/or the first marker comprised by the image, a position and/or orientation of the patient couch relative to the medical facility, wherein for positioning the mobile patient couch, the drive and/or the steering unit is activated on the basis of the position and/or orientation determined.

The starting point below, without restricting generality, is a patient as the examination object, wherein this mostly involves a human being. Basically the patient can also be an animal. The two terms “examination object” and “patient” are used synonymously below. As an alternative, the examination object can be a plant or an inanimate object, for example a historic artifact or the like.

The method relates to in particular a mobile patient couch, which is capable of positioning itself relative to a stationary medical facility, such as for example a tomography device. This couch is conceived to move in various directions and to orient itself to specific positions. The method and/or the patient couch in particular makes provision for it to be able to be controlled by a user, for example by specifying start point and end point. The patient couch can further form an autonomously drivable patient couch, which is able to be controlled for example by a control facility.

The patient couch comprises a control facility, which is connected for exchange of data with a drive and/or a steering unit, wherein the control facility can control and/or regulate the drive and/or the steering unit. Preferably the control facility can control the drive for braking, acceleration and/or maintaining a speed and/or the control facility controls the steering unit for steering the patient couch. The control facility receives and processes control signals in order to check the movements of the couch. The drive and the steering unit make possible precise movements and positionings of the couch within the medical facility.

The drive and/or the steering unit of the patient couch are designed so that they can be controlled based on the control signals received by the control facility. This allows a precise control and adaptation of the movements of the couch in order to guarantee accurate positioning.

### POSSIBLE EMBODIMENTS OF THE DRIVE AND THE STEERING unit can comprise:

Chassis: The couch can be equipped with a robust chassis, which makes possible a stable and even movement. The chassis can have springing systems, which minimize shocks and vibrations in order to enhance the comfort of the patient.
Electric drive: The use of electric motors for the drive of the couch can guarantee a quiet and efficient locomotion. Electric motors moreover offer the advantage that they can be precisely controlled and regulated, which is decisive for the exact positioning of the couch.
Steering system: The steering unit can be realized by an electrical or hydraulic steering system. An electrical steering system could be sensor-based and make automatic adjustment of the steering movements possible. A hydraulic steering system on the other hand could excel with its robust construction and operate reliably in particular in in challenging environments.

The patient couch is furthermore equipped with an optical sensor facility, which can be a camera, for example. This sensor facility is designed to capture images of the environment of the camera. The camera is in particular arranged so that a floor and/or floor area is able to be captured. The camera is in particular arranged so that it can capture an area lying in a longitudinal direction in front of it. For example the camera has a capture direction, wherein the capture direction encloses an angle with a horizontal direction and/or the forwards direction of the patient couch of −10 degrees and −80 degrees, preferably an angle of −30 degrees and −60 degrees.

The optical sensor facility captures at least one image, in particular images. The image shows an environment of the camera and/or the medical facility. The image in this case shows the first optical marker. The first optical marker is arranged in the environment of the medical facility, in particular in the floor area. The at least one optical marker serves in particular as a reference for the positioning of the couch.

The sensor facility captures then first optical marker in an image and makes this image available to the control facility. This makes it possible for the control facility to analyze the first optical marker and to determine the position relative to it, in particular to the medical device. The image can be a color image, a grayscale image and/or black and white image. In particular the optical sensor facility can be embodied to capture and provide a stream of images and/or video comprising the image.

Based on the image of the first marker provided and/or the first marker comprised by the image, the control facility determines the position and/or orientation of the patient couch relative to the medical facility. This makes sure that the couch can be precisely aligned and positioned. In particular the control facility, based on the image of the first marker and/or the first marker comprised by the image determines an actual position and/or actual orientation, wherein the control facility, based on the actual position and/or the actual orientation and a required position and/or required orientation, can control the drive and/or the steering unit so that the patient couch reaches and/or assumes the required position and/or required orientation.

For positioning the mobile patient couch the drive and/or the steering unit is controlled based on the position and/or orientation determined. This guarantees an exact positioning of the couch, so that the patient is aligned optimally for the medical examination. The control facility, the drive, the steering unit and the optical sensor facility work synergistically with each other to make possible a precise and reliable positioning of the patient couch. The control facility analyzes the images acquired by the sensor facility and sends corresponding control signals to the drive and/or the steering unit in order to move and align the couch.

Through the integration of the optical sensor facility and the first optical marker, the position and orientation of the patient couch can be determined with high accuracy. This makes possible a more precise positioning of the patient relative to the medical facility, for example a medical imaging system, and/or within the examination volume of the medical imaging system.

The described method and the associated patient couch offer significant advantages for medical facilities, since they significantly improve the positioning and alignment of the patient for diagnostic imaging methods such as tomography or magnetic resonance tomography. This leads to a higher accuracy of the diagnostic images and improves the comfort and the security of the patient during the method as a whole.

The patient couch can have a docking facility, wherein the docking facility can interact with a docking point of the medical facility. Through the docking system the patient couch is connected mechanically, electrically and/or for exchange of data with the medical facility, for example a medical imaging system, and achieves an exact positioning of the patient couch, so that a patient who is supported on a stretcher board of the patient couch, can be positioned to the precise millimeter in the examination volume of the medical imaging system. Plug-in locations on the patient couch, for example for local coils with a magnetic resonance system, are usually connected via cables to the docking system or the patient couch side docking facility, so that measurement signals and/or send signals obtained can be transported to the medical imaging system or received from said system.

The mobile patient couch, as well as the drive and/or the steering unit, can comprise a chassis, in particular drive and/or steering unit can be part of the chassis. The chassis of the patient couch comprises a plurality, i.e. at least two, preferably at least four wheels, especially preferably more than four wheels. The wheels are embodied to make possible a locomotion of the patient couch, wherein the wheels are preferably able to be driven by the drive and/or able to be steered by the steering unit.

Preferably the four wheels are arranged in corner locations, i.e. at the corners of the carrier device, in order to achieve the best possible stability of the patient couch. If the patient couch is pushed for example in a manual movement brought about by a user for example via a couch grip arranged transverse to the longitudinal axis of the couch, the casters are advantageously aligned according to the manual directional specification and the patient couch travels in the desired direction.

The inventive method serves to position a mobile patient couch, in particular relative to a stationary medical facility. The patient couch is embodied to support a patient. In particular the mobile patient couch is an autonomously driven and/or steerable patient couch. The patient couch preferably comprises a drive and/or a steering unit. The drive is embodied to drive, to accelerate and/or to brake the mobile patient couch. The steering unit is embodied to steer the patient couch and/or wheels of the patient couch.

The patient couch comprises a control facility. This control facility can form a hardware or a software module. The control facility is embodied to control the drive and/or the steering unit of the patient couch, to regulate it and/or to supply it with control signals. The mobile patient couch preferably forms an autonomous, drivable and/or steerable patient couch. The mobile patient couch can further form a hybrid patient couch, which can be operated both autonomously also under user control. For example the user can independently control, steer and/or regulate the speed of the patient couch in unproblematic areas, and on the other hand operate it in an autonomous operating mode in more complex situations.

The method serves in particular for positioning the mobile patient couch relative to a stationary medical facility.

The stationary medical facility can for example be an imaging device, specifically a computed tomograph, an x-ray facility (also referred to as an x-ray device) or a magnetic resonance tomograph. As an alternative and/or in addition, the stationary medical facility can form a treatment facility (also referred to as a treatment device), for example a linear accelerator for radiation therapy of a patient or another type of treatment facility. The stationary medical facility is arranged at a fixed place within an environment and/or a room.

The method makes provision for the mobile patient couch to be positioned automatically and/or autonomously in a required position and/or required orientation relative to the stationary medical facility. For example the mobile patient couch is subsequently to be coupled to the stationary medical facility. The method positions the mobile patient couch so that it is arranged relative to and/or at the stationary medical facility, so that this can be coupled and/or necessary examinations and/or treatments can be carried out. The method makes provision for the positioning to be undertaken as comfortably, safely and quickly as possible for the patient.

The mobile patient couch comprises at least one optical sensor facility. Specifically the patient couch preferably comprises at least two optical sensor facilities. As well as the optical sensor facilities the patient couch can comprise further sensor facilities, for example distance measuring devices, collision warning systems and also acceleration and/or velocity sensors. The optical sensor facility comprises and/or forms a camera for example, in particular a color camera. The optical sensor facility and/or the camera has sensor facility parameters and/or camera parameters, which for example describe the imaging and or capture parameters of the optical sensor facility. The sensor facility and/or the control facility are preferably provided with the sensor facility and/or camera parameters.

The optical sensor facility is embodied to capture and/or to record a sensor facility environment. In particular the optical sensor facility is embodied to capture at least one image, a plurality of images, an image sequence and/or a video of a camera environment. The camera environment is in general also to be understood as a sensor facility environment. The sensor facility environment is the area that can be captured and/or shown by the sensor facility in an image. Preferably the optical sensor facility is arranged so that the camera environment able to be captured is oriented in the direction of travel and/or floor direction. In particular the optical sensor facility is embodied to capture a floor area and/or an area in direction of travel or forwards direction as an image.

The medical facility has an environment; wherein first optical markers are arranged in this environment. When optical markers are referred to here and below, this is to be understood in the singular as one optical marker and also as a plurality of optical markers. Specifically the method is able to be executed provided an optical marker, an optical second marker and/or an optical further marker is captured by the sensor facility and evaluated by the control facility. In particular at least one optical marker is arranged in the environment of the medical facility. The environment of the medical facility, in which the marker or the first optical markers is or are arranged, is preferably located in front of the medical facility, wherein “in front of the medical facility” in particular describes the area at which the mobile patient couch is to be positioned and/or is arranged in a coupled state. The environment of the medical facility, in which the marker or the optical markers are arranged, is located in particular at a distance of less than one meter, preferably of less than 0.5 meters, away from the medical facility.

The sensor facility records at least one image, a number of images, an image sequence and/or a video of the camera environment, wherein at least one of these images, number of images and/or the image sequence and/or the video, captures the floor area in the environment of the medical facility. In other words at least one image shows the optical marker or markers that are arranged in the environment of the medical facility, specifically in the floor area in the environment of the medical facility. Preferably at least one image shows the marker or the entire optical markers, as an alternative an image shows at least 50 % of the optical marker or markers. The optical sensor facility is embodied to provide at least one, a plurality or all images obtained, which show the floor area in the environment of the medical facility and/or the first optical marker or markers, to the sensor facility. The images are provided in particular as image data, for example as an image stream.

The at least one image shows the optical marker and/or the optical markers captured by the optical sensor facility, in particular from the position and/or perspective of the optical control facility, in particular of the camera. The optical marker and/or the optical markers shown in and/or comprised by the image can appear changed compared to their actual geometry, shape and/or size, in particular stretched, squashed, rotated, distorted and/or misrepresented. In particular the changed appearance is based on the sensor facility parameters and/or the camera parameters.

The control facility is provided with the image and/or the images, in particular in the form of image data. The control facility is embodied to evaluate the image and/or the images, which show the floor area in the environment of the medical facility and/or the first optical marker or markers and/or to employ them for the control of the mobile patient couch, in particular of the drive and/or the steering unit.

In particular the control facility can be embodied to process the images provided in a pre-processing, for example to enhance or reduce the image quality, for optimization of the evaluation areas and/or for reduction of image errors.

Based on the image and/or images provided which show the marker or markers, specifically based on the first marker or markers comprised by the image and/or the images, the control facility controls and/or regulates to determine a position and/or an orientation of the patient couch, in particular relative to the medical facility and/or relative to the optical first markers. For example the control facility, based on the control facility parameters and/or the camera parameters as well as the image provided and/or the images provided, determines a relative position and/or orientation as regards the optical marker and/or the medical facility. This determination can for example be based on how the relative position and/or orientation must be so that the recording of the optical marker or markers shown in the image or the images correspond to the representation of the optical first marker. Based on this determined position, location and/or orientation, the control facility controls the drive and/or the steering unit. In particular the steering unit and/or the drive is controlled so that the mobile patient couch approaches the environment and/or the floor area, in particular the first optical marker.

In an optional embodiment at least one second optical marker, in particular a number of second optical markers, is provided. The marker or the second optical markers can in particular be embodied similarly to the first optical marker. For example they have the same geometrical form and/or the same structure. The second optical markers are preferably smaller than the first optical markers, in particular at least half as large, specifically at least 10 % as large. The second optical markers and/or the second optical markers are arranged on the medical facility. In particular they are fitted in the front area of the medical facility. Specifically the second optical markers and/or the second optical marker are placed in the lower area of the medical facility, wherein the lower area for example describes a lower third of the medical facility. Preferably the second optical markers are in the vicinity of the first optical marker or align with these.

The sensor facility is embodied to record or to capture at least one image or a plurality of images of the second optical markers. Specifically the sensor facility can be embodied so that in an image both the first and also the second optical markers are captured and provided to the control facility. In this case the sensor facility is arranged so that, on approach of the patient couch for reaching the required position and/or orientation, both the first and also the second optical markers are captured. The sensor facility is embodied to provide the image comprising and/or showing the second optical markers and/or the second optical marker, to the control facility. Just as for the first optical markers, the second optical markers are shown in the image just as they will be recorded and/or imaged from position, orientation or perspective and corresponding to the sensor facility parameter. Based on the knowledge about the sensor facility parameters, the control facility can determine a relative position and/or orientation of the patient couch to the second optical markers and thus to the medical facility.

While the first optical markers are preferably used for rough positioning and/or orientation of the patient couch, via the second optical markers a fine position and/or fine orientation of the patient couch relative to the medical facility and/or relative to the optical markers can be determined. The control facility is embodied, based on the fine position and/or fine orientation determined, to control the drive and/or the steering unit of the patient couch for positioning. Specifically, the drive and/or the steering unit are activated based on the position and/or orientation determined via the first markers and the fine position and/or fine orientation determined via the second markers. It is especially preferred for the control facility, based on an image that shows the first and second optical marker, to determine the position, fine position, orientation and/or alignment and, based thereon, to control the drive and/or the steering unit.

This embodiment allows a precisely automated positioning of the patient couch and one that is comfortable for the patient to be made possible. In particular, through the arrangement of the first and second markers provided a low level of disturbance to the environment is provided, which in particular has a less disruptive or disturbing effect on personnel and/or patient.

In an especially preferred embodiment there is provision for the control facility to control the patient couch, in particular the drive and/or the steering unit, differently in a far area and in a near area, in particular based on different information, data and/or fundamentals. The far area is for example an area at a distance of greater than 1.5 meters, specifically greater than 2 meters, from the stationary medical facility, from the required position and/or from the floor area in which the first markers are arranged. The near area is preferably an area at a distance of less than 1.5 meters, specifically of less than 1 meter, to the floor area in which the first markers are arranged, and/or from the required position and/or from the stationary medical facility. In this case there can be provision for the control facility to control the drive and/or the steering unit of the patient couch in the far area based on the position and/or determined orientation on the basis of the first markers. Further there is preferably provision in this case for the control facility to control the steering unit and/or the drive in the near area based on the fine position determined and/or the fine orientation determined, specifically based on the position and/or orientation determined via the first markers and based on the fine position and/or fine orientation determined by the second markers. In other words there is provision for example for the control of the drive and/or the steering unit by the control facility to be undertaken in the far area without fine position and/or fine orientation, in particular without recourse to the evaluation of the second markers. This embodiment makes possible an especially efficient, fast and precise control.

It is especially preferred for the control facility to be embodied to determine a trajectory for the mobile patient couch. The trajectory describes for example the path, in particular the settings such as velocity and/or acceleration, to the stationary medical facility, in particular for positioning relative to and/or at the medical facility. The control facility determines the trajectory preferably based on the image and/or the images that show the first markers and/or the second markers. Based on the first and/or second markers shown in the image and with knowledge of the sensor facility parameters, the trajectory is determined. In particular the trajectory runs from an actual position to a required position. The required position is in particular the position in which the mobile patient couch is to be positioned and/or oriented. Preferably there is provision for the control facility to determine the trajectory so that it describes a shortest, most even, least curving and/or most patient protective path. In other words the trajectory is preferably determined so that the fewest possible shocks, lateral accelerations and/or longitudinal accelerations occur and/or no objects in the environment are touched. Especially preferably the required position is a position in which the patient couch is docked at the stationary medical facility. The control facility is embodied to control the drive and/or the steering unit, in particular the patient couch, based on the trajectory determined, so that the patient couch arrives at the required position and/or required orientation and/or is positioned there.

In an optional embodiment of the present invention there is provision for the at least one further optical marker to be provided. Further optical markers in the plural will generally be referred to below. The further optical markers are preferably similar or identical in structure, in form and/or type to first and/or second optical markers. The further optical markers are for example arranged on a wall, on walls and/or on objects and/or other items in the environment of the medical facility. In particular the further optical markers are placed close to the floor on the wall or the walls and/or the objects. The sensor facility is embodied to detect the further optical markers in an image. For example individual or a number of optical markers, also the further optical markers, are also captured in an image during capturing of the first and/or second optical markers. In other an image captured and provided to the control facility can show and/or map the first, second and further optical markers. The control facility is embodied, based on the image that comprises the further optical markers, to determine environment information. For example the environment information is encoded in and/or comprises the further optical markers. The environment information can for example contain information about space, floor, room or other environment information. In particular the environment information can also comprise information regarding the medical facility and/or regarding obstacles in this area. The control facility is embodied, based on the environment information and/or the image of the further markers provided, to control the steering unit and/or the drive of the patient couch.

It is especially preferred that the first, the second and/or the further optical markers form an optical code. For example the optical code forms a there and/or code. The optical markers in this case are embodied and/or constructed for example according to a predetermined pattern and/or structure. The optical markers and/or the optical code comprise and/or encode marker information. In other words the first, second and/or further optical markers each comprise marker information, wherein this marker information is encoded into the optical markers. In particular the marker information that is encoded in the optical markers differs between the individual optical markers. The marker information can for example comprise information about the marker type, for example first marker, second marker or further marker. As an alternative and/or in addition the marker information, it can comprise information regarding position, orientation, medical facility and/or other information needed for determination of the trajectory or for positioning of the patient couch. The optical markers in particular form flat and/or two-dimensional optical markers. Especially preferably the first, second and/or further optical markers, referred to for short below just as optical markers, form a regular geometrical figure and/or have a regular geometrical form. For example the optical markers have a square, rectangular, triangular or polygonal form.

The optical markers preferably comprise a plurality of marking elements. Preferably the number of marking elements is equal to or is limited by a maximum number. Preferably the marking elements define the geometrical form of the optical markers and/or are defined by the geometrical form in their arrangement. For example a definition of the marking element positions is given by the regular geometrical form of the optical markers at which marking elements are arranged and/or are possible. Thus the absence of a marking element at such a required position can contribute to the encoding in the optical code. Preferably an optical marker comprises exactly or at least four marking elements. The marking elements in this case have a simple geometrical form and/or form a simple geometrical figure, for example a circle, rectangle and/or a regular polygon.

Specifically there can be provision for all marking elements to form an identical geometrical figure. As an alternative, marking elements within an optical marker can differ in their geometrical figure and/or form. The marking elements are positioned and/or arranged according to an arrangement scheme in the geometrical form of the optical marker, for example these are preferably arranged at corners of the geometrical form. Through the type and/or arrangement of the marking elements the marker information is encoded in the optical marker. The capture of an optical marker in an image enables the control facility to decode the marker information and include it for controlling, in particular positioning, the patient couch.

In an especially preferable embodiment there is provision for the optical marker to form and/or have a regular geometrical form, in particular a rectangle and/or a square. The regular geometrical form has corner areas, in particular corresponding to the number of corners of the geometrical figure. In this case there is preferably provision for the marking elements to be arranged in these areas and/or in places at which they are arranged in particular so that in the intervening areas no marking elements are to be expected and/or provided. The arrangement of the marking elements in the corner areas enables the geometrical form of the optical markers to be defined and/or fixed, so that by evaluation of the geometrical form in the captured images, a relative position and/or orientation is able to be determined by the control facility. In particular through the definition of the arrangement of the marking elements in corner areas, the absence of a marking element can also be used for encoding, provided the regular geometrical form of the optical marker is still uniquely defined by the other marking elements present.

It is especially preferred that the marking elements differ in part in their form, size and/or color. For example the difference in the form of the marking elements, the difference in the size and/or the difference in the color of the marking elements used enables the marker information to be encoded. For example circles, triangles and/or rectangles can be used as marking elements to differentiate between the marking elements. The marking elements can further differ in their coloring or their form. Specifically this can also be implemented by color-filled marking elements or by an outline and non-color-filled marking elements. The colors are preferably differentiated in the RGB color model. As an alternative and/or in addition use can be made of different size marking elements for encoding the marker information.

There is optional provision for the control facility, based on the image provided of the first, second and/or further markers, in particular of first, second and/or further marker, comprised by the image, to determine the marker information encoded in the markers. In this case the control facility, for positioning the mobile patient couch, can control the drive and/or the steering unit based on the marker information and/or determine the trajectory based on the marker information. In other words the control facility can include the encoded information in the markers, the marker information for positioning, control and/or trajectory determination. For example the marker information can comprise information about obstacles, about offset parameters and/or positioning tips.

It is especially preferred for the optical markers to be embodied larger than the second and/or further optical markers. In particular the first optical markers are at least twice, specifically at least ten times as large as the second or further optical markers. This is based on the consideration that the first optical markers are arranged in a floor area and are employed by the sensor facility for first rough positioning and/or orientation of the patient couch and the second and/or further optical markers are only employed later for fine positioning and/or fine orientation. It this case it is to be expected during fine positioning and/or fine orientation, that the patient couch and thus the sensor facility is already located closer to the medical facility and smaller optical markers can be employed for capture and evaluation. Such optical markers are felt to be less disruptive and are also able to be attached in smaller areas and/or without restriction to the medical facility. Larger optical markers in the floor area like the first optical markers are likewise felt to be less disruptive, since due to the dimensioning using simple marking element, their function as markers will not be deduced automatically by the patient.

It is especially preferred for the first and the second optical markers to be arranged flush with regard to a longitudinal direction of the patient couch in the required position, in particular in the positioned and/or docked status. In other words, when viewed in the longitudinal direction of the patient couch, when said couch is in the required orientation and/or required position, the first and the second markers are in a line. This is based on the consideration that, during an approach of the patient couch already in the required orientation to the medical facility, both the first and also the second marker can be captured. In particular, based on the relative arrangement of the first and the second optical marker, the trajectory and/or the positioning of the patient couch can be determined better and more accurately.

A patient couch forms a further subject of one or more example embodiments of the present invention, wherein the patient couch has a steering unit and/or a drive. The patient couch in particular forms a motorized and/or at least partly autonomously driven patient couch. The patient couch has a control facility, wherein the control facility is embodied to control the steering unit and/or the drive of the patient couch for positioning of the patient couch.

The patient couch further has a sensor facility. The sensor facility forms an optical sensor facility and comprises a camera for example. The sensor facility is embodied to capture images of a camera environment, in particular images, comprising and/or showing a first optical markers, second optical markers or further optical markers. The sensor facility is embodied to provide the captured images, in particular a captured image comprising and/or showing the optical first markers, optical second markers and/or further optical markers, to the control facility.

The control facility is embodied in this case, based on the image provided, showing the first, the second and/or third markers, to control the patient couch, the drive and/or the steering unit, for example by determining a relative position and/or relative orientation of the patient couch to the medical facility, to the optical first, second or further markers. In particular the patient couch, the sensor facility and/or the control facility are embodied to carry out the inventive methods described above. In other words the patient couch implements and/or makes possible the inventive method.

It is especially preferred that the patient couch comprises and/or has a camera as its sensor facility. The camera is embodied for capturing the image and/or for capturing images. The camera has an acquisition direction, for example a capture or viewing direction of the camera. The camera and/or the sensor facility is arranged so that the capture direction points forwards in the direction of travel. In particular the camera and/or sensor facility are arranged with their capture direction tilted downwards, so that at least on part of a floor area is captured. This arrangement makes it possible for both first markers arranged in the floor area and also second markers arranged on the medical facility to be recorded and/or captured by the camera.

A positionable arrangement forms a further subject of one or more example embodiments of the present invention. The positionable arrangement comprises the patient couch, in particular the inventive patient couch, as well as a stationary medical facility, for example in the form of an imaging facility or a treatment facility. First optical markers as described previously are arranged in a floor area in an environment of the medical facility. Second optical markers can further be arranged on the medical facility. The patient couch, in particular the sensor facility, is embodied to capture at least one image and provide it to the control facility, wherein this image shows and/or maps the first optical markers and/or second optical markers. The patient couch and/or the control facility and/or the sensor facility is embodied to make possible and/or to implement the inventive method.

### BRIEF DESCRIPTION OF THE DRAWINGS

Further advantages, effects and embodiments emerge from the enclosed figures and from their description. In the figures:

FIG. 1 shows an arrangement comprising a mobile patient couch and a stationary medical facility;

FIG. 2a and FIG. 2b show a further arrangement comprising a mobile patient couch and a stationary medical facility;

FIG. 3a and FIG. 3b show a further arrangement comprising a mobile patient couch and a stationary medical facility;

FIG. 4a and FIG. 4b show a further arrangement comprising a mobile patient couch and a stationary medical facility;

FIG. 5 shows two (coordinate) systems;

FIG. 6 shows a few examples of optical markers;

FIG. 7 shows a block diagram for a method for positioning a patient couch.

### DETAILED DESCRIPTION

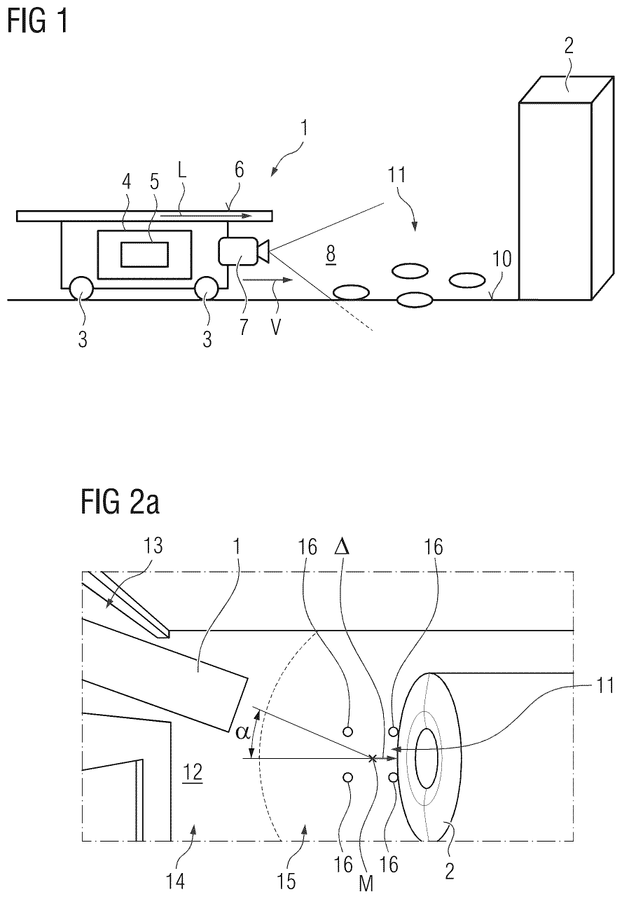

FIG. 1 shows an arrangement comprising a mobile patient couch 1 and a stationary medical facility 2 (also referred to as a stationary medical device) for carrying out the method for positioning the mobile patient couch. The mobile patient couch 1 comprises wheels 3, which can be driven and controlled by a steering unit and/or a drive 4. The steering unit and/or the drive 4 are connected to a control facility 5 (also referred to as a control device or controller), so that the control facility 5 can control the drive 4 and/or the steering unit via control signals. The control facility 5 can be a hardware or a software module.

The patient couch 1 comprises a stretcher board 6, on which a patient can be supported. The stretcher board 6 in particular defines a longitudinal direction L of the patient couch 1, which for example extends from a foot area to a head area. The patient couch 1 further defines a forwards direction V. The forwards direction V is in particular equated to the longitudinal direction L.

The mobile patient couch 1 comprises an optical sensor facility 7 (also referred to as a optical sensor or optical sensor device). This is embodied here as a camera or comprises a camera. The optical sensor facility 7 has a data link and/or a signaling link to the control facility 5. The optical sensor facility 7 has a capture area 8, which points at least partly in the forwards direction V. Specifically the optical sensor facility 7 can be arranged so that the capture area 8 is tilted towards the floor. The optical sensor facility 7 is embodied to record at least one image 9 of the capture area 8.

In the capture area 8 for example a part of or the entire stationary medical facility 2 is arranged and thus comprised or shown in the image 9.

Arranged in the floor area 10, which is located between the patient couch 1 and the stationary medical facility 2, is a first optical marker 11. This marker 11 is thus arranged in the capture area 8 and is captured by the optical sensor facility 7. The optical sensor facility 7 thus captures an image 9, which shows the optical first marker 11 and part of the stationary medical facility 2.

The captured image 9 is provided to the control facility 5. In other words the image 9, which shows the optical marker 11 as seen by the optical sensor facility 7 will be provided to the control facility 5. The control facility 5 is embodied, based on the image 9 provided and knowledge about form, size and/or structure of the optical marker 11, to determine a position and/or orientation of the patient couch 1 relative to the medical facility 2.

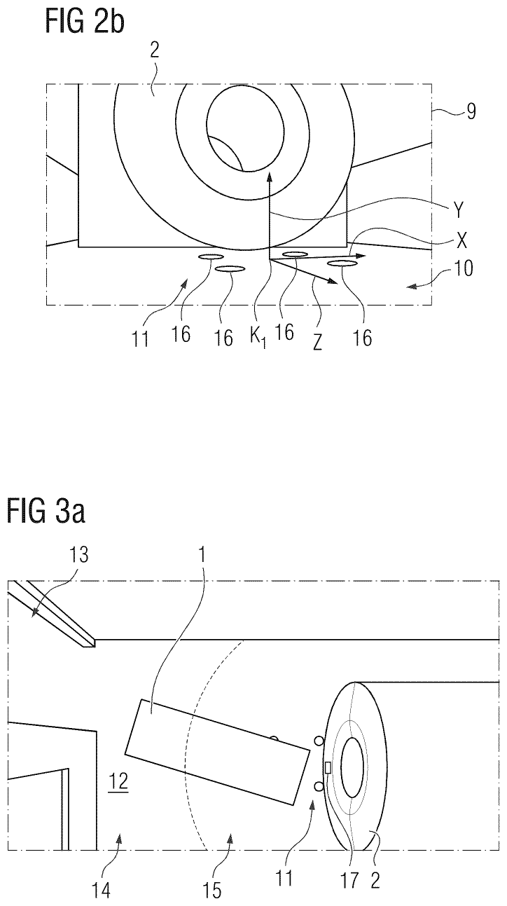

FIG. 2a shows a similar arrangement of a mobile patient couch 1 and a stationary medical facility 2 for carrying out the method for positioning of the patient couch 1 in a view from above. The stationary medical facility 2 is located in a room 12, also called the examination room.

The room 12 is accessible via a door 13. A patient, who is to be examined with the stationary medical facility 2 can be brought via the mobile patient couch 1 to the medical facility 2. To this end the patient couch 1 can be moved through the door 13.

For positioning the patient couch 1 and thereby the patient at the medical facility 2, in particular for docking the patient couch 1 at the medical facility 2, the method of one or more example embodiments of the present invention can be employed.

In FIG. 2a the mobile patient couch 1 is located in a far area 14 away from the stationary medical facility. The far area 14 is for example an area that is at a distance of more than 1.5 m from the stationary medical facility. A near area 15 further exists, which for example is less than 1.5 m away from the stationary medical facility 2.

In the arrangement from FIG. 2a shown, the optical sensor facility 7 captures the first optical marker 11, which is arranged on the floor. The first optical marker 11 here comprises four marking elements 16. The optical sensor facility 7 thus captures an image 9 that shows the first optical marker 11 or the marking elements 16. Based on this image 9, on known optical parameters of the sensor facility and on the knowledge about form, geometry, size and/or structure of the marker 11, the control facility 5 determines a relative orientation and/or position of the patient couch 1 in relation to the stationary medical facility 1. To do this, the control facility can for example determine an angle α, which describes the angle enclosed between a required alignment and the forwards direction V. Form, geometry, size and/or structure of the marker 11 can for example be understood as the knowledge about the form in which the marking elements 16 are arranged, for example quadratically, and/or form, color and/or distances between the marking elements 16.

The control facility 5 can further be embodied to determine a distance d that describes the distance of the patient couch 1 from a midpoint M of the marker 11. Based on the offset Δ between midpoint M and stationary medical facility 2 and also the distance d, the relative position and/or orientation can be determined by the control facility 5.

FIG. 2b shows an image 9 captured by the optical sensor facility 9. The image 9 shows the floor area 10, the stationary medical facility 2 and the first marker 11 from the perspective of the optical sensor facility 7. The optical marker 11, in a view from above, is a quadratic figure, wherein marking elements 16 are each arranged in its corners. In the image 9 the first marker is not shown quadratically, but is distorted. Based on imaging parameters of the sensor facility 7 and the knowledge about the structure of the marker 11, the relative orientation and/or position can be determined. To do this, a reference system K1 can be employed by the optical marker 11, which comprises the axes X, Y and Z. For example the origin of this reference system K1 can be set in the midpoint M. The axis Z here defines a vertical direction.

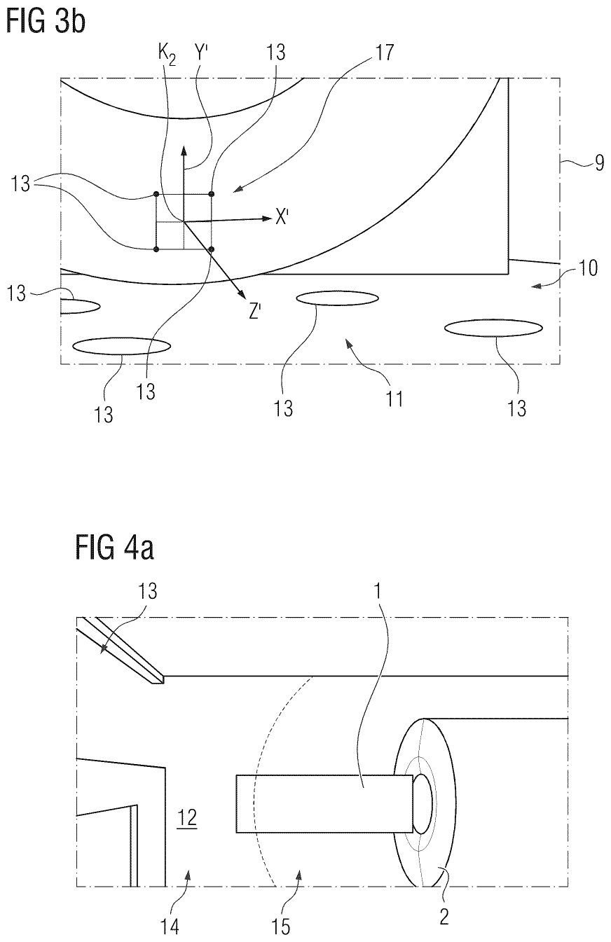

FIG. 3a shows the arrangement of patient couch 1 and medical facility 2 from FIG. 2a, wherein here the patient couch 1 is located in the near area 15. Arranged on the stationary medical facility 2 on a front side is at least one second optical marker 17. Here the second optical marker 17 also comprises four marking elements 16, which are arranged quadratically. Based on the arrangement of the marking elements 16 a reference system K2 can be defined, which comprises the axes X′, Y′ and Z′.

The optical sensor facility 7 is embodied to record at least one image 9.

The image 9 (FIG. 3b) shows the second optical marker 17 from the perspective of the optical sensor facility 7. From the perspective of the optical sensor facility 7 the four optical marking elements 16 do not form a square but rather a distorted polygon. On the basis of the optical parameters of the sensor facility 7 and the knowledge that the marking elements 16 are arranged quadratically, a relative orientation and/or position of the patient couch 1 can be determined by the control facility 5. In the near area 15 the position and/or orientation are determined as fine position and/or fine orientation, which is more precise than the determination of the orientation and/or position in the far area 14. The control facility 5 can control the patient couch 1, in particular the drive, based on the position, orientation, fine position and/or fine orientation determined. For example, based on the positions and/or orientations determined, a trajectory is determined that guides the patient couch to a required position and/or required orientation.

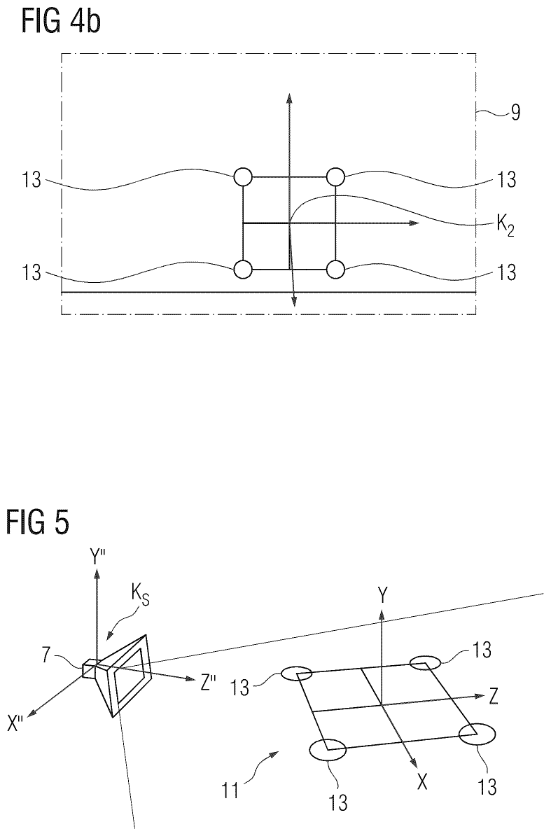

FIG. 4a shows the arrangements from FIG. 2a and 3a, wherein here the patient couch 1 is arranged in its required position and/or required orientation relative to the stationary medical facility 2. The required position and/or required orientation is intended for the patient couch 1 with its longitudinal direction L and/or forwards direction V to be in the same alignment for an insertion direction into the medical facility 2. Here the patient couch 1 and the stationary medical facility 2 can be coupled, for example via interfaces.

FIG. 4b shows the second optical marker 17 together with the reference system K2 and the axes X′, Y′ and Z′. The optical marking elements 13 of the second marker 17 are arranged in image 9 and thus from the perspective of the optical sensor facility 7 in a square and thus not distorted. A sensor system KS can further be defined in image 9 and/or for the sensor facility 7, which comprises the axes X″, Y″ and Z″. If the patient couch 1 is located in its required position and/or in the coupled or docked state, the axes X, X′, X″, Y, Y′, Y″ and also Z and Z″ are aligned in parallel and/or are the same.

To illustrate the different systems K1, K2, KS a first marker 11 with marking elements 13 and axes X, Y and Z is shown in FIG. 5. The optical sensor facility 7 with its capture area defines the System KS the axes X″, Y″ and Z″. Depending on position and orientation of the sensor facility 7 on the marker 11, the systems K1 and KS are twisted and/or tilted in relation to each other. Based on the imaging parameters of the sensor facility 7 and the knowledge about the structure of the marker 11, the orientation between the systems K1 and KS can be determined by the control facility 5.

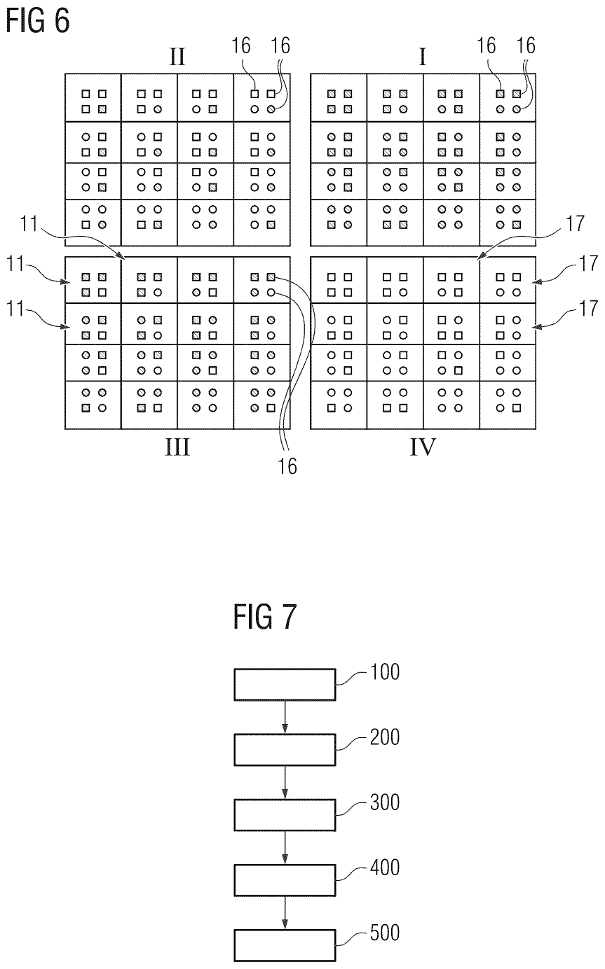

FIG. 6 shows different variants of optical markers, which can be used as first markers 11, second markers 17 or further optical markers. These markers serve as encoding elements and encrypt information, such as for example marker information. The encoding is based in particular on the use of different marking elements 13. For clarity and illustration of the structure, the different markers and codes in FIG. 6 are divided into four quadrants I, II, III and IV.

The optical markers are essential elements for precise positioning and alignment of the patient couch 1 with regard to the medical facility 2. Each optical marker comprises four marking elements 13, which are arranged in the corner of a square.

The marking elements 13 are arranged in a square, wherein each element 13 is placed at one of the four corners. This arrangement makes possible a clear definition of the marker structure and supports the correct capture by the optical sensor facility 7.

Various forms and colors of the marking elements 13 can be used for encoding of information and for definition of the orientation of a marker. In the present exemplary embodiment the marking elements 13 are distinguished in their form—they are either circular or square—and in their color. The colors of the marking elements in this case are especially important and are preferably defined and measured on the HUE scale. This scale is advantageous for encoding and decoding by comparison with the RGB scale. In this system preferably two different colors are used, namely orange and turquoise. This choice of color is based on specific requirements such a branding, marker concept, design or corporate identity in order to minimize visual disruptions.

Through the use of different forms and colors each of the four positions in a marker offers four possible combinations. This leads to a total of 4×4×4=256 possible combinations, which corresponds to an 8-bit encoding. This diversity makes possible an efficient and precise transmission and processing of information within the system.

Shown in quadrant I are markers that are intended to be fixed to a wall, in particular in the floor area. These markers encode information about the respective room, such as for example a room number, room type or other room-specific data. The markers can for example form further markers in accordance with one or more example embodiments of the present invention. The control facility 5 can check with the aid of this information whether it is in the right room. Moreover it can load a plan of the environment in order to recognize and to take account of obstacles.

Quadrant II shows markers that are intended to be fixed to the ceiling. These markers can comprise information about devices suspended from the ceiling, such as for example C-arms. Such information is important since these devices can represent potential obstacles. The control facility 5 can use this data to adapt the navigation of the patient couch 1 accordingly.

Shown in quadrant III are first optical markers 11 for attaching in front of the medical facility 1 and/or to the floor. These markers 11 can for example encode an offset Δ, i.e. the distance between the middle of the optical marker 11 and the medical facility 2. It can furthermore encode information about the stationary medical facility 2 itself, such as the type of facility (for example CT or MRT). This information is used by the control facility 5 for precise positioning and orientation of the patient couch 1 relative to the medical facility 2.

Quadrant IV shows second optical markers 17, which are attached to the medical facility 2. These markers 17 can comprise information for coupling, such as interfaces, or for more precise positioning of the patient or of the patient couch 1. The height of the bore of an MRT or CT or other mechanical interfaces can be encoded. This data is decisive for the precise alignment and docking of the patient couch 1 at the medical facility 2.

Shown in FIG. 7 is a block diagram, which describes the method for positioning a mobile patient couch 1. The method comprises the steps:
Step 100: Capturing of the environment. The optical sensor facility 7 of the patient couch 1 captures images of the camera environment. First optical markers 11, which are arranged in the floor area 10 around the medical facility 2, can be seen in image 9.
Step 200: Image processing. The sensor facility 7 forwards the image captured to the control facility 5. This analyzes the image 9 in order to determine the position and/or orientation of the patient couch 1 relative to the medical facility 2, based on the markers 11 captured.
Step 300: Position determination. The control facility 5 computes the position and/or orientation of the patient couch 1 with the aid of the information contained in the image 9. In this case the relationship between the markers 11 and the patient couch 1 is taken into account.
Step 400: Control of the movement. Based on the computed position and orientation data, the control facility 5 controls the drive and/or the steering unit 6 of the patient couch 1 in order to position said couch precisely.
Step 500: Refinement and adjustment. If necessary, further corrections are made. The sensor facility 7 can furthermore capture images and send them to the control facility 5 in order to guarantee an ongoing adjustment of the position and orientation of the patient couch 1. This can be undertaken for example based on the second optical markers or further optical markers.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A method for positioning a mobile patient couch relative to a stationary medical device, wherein the mobile patient couch includes at least one of a controller, a drive unit or a steering unit, wherein at least one of the drive unit or the steering unit is configured to be controlled by the controller based on control signals, wherein the mobile patient couch includes an optical sensor configured to capture at least one image of a camera environment, wherein at least one first optical marker is arranged in a floor area in an environment of the stationary medical device, and wherein the method comprises:
capturing, by the optical sensor, the at least one first optical marker in an image;
providing the image to the controller;
determining, by the controller, at least one of a position or an orientation of the mobile patient couch relative to the stationary medical device, based on the image of at least one of the at least one first optical marker provided or the at least one first optical marker comprised by the image; and
positioning the mobile patient couch by controlling the at least one of the drive unit or the steering unit based on the at least one of the position or the orientation.

2. The method as claimed in claim 1, wherein
at least one second optical marker is arranged on the stationary medical device; and
the method further includes
capturing, by the optical sensor, the at least one second optical marker in an image, and
providing the image including the at least one second optical marker to the controller, wherein
the controller provides at least one of a fine position or a fine orientation of the mobile patient couch relative to the stationary medical device based on at least one of the image including the at least one second optical marker provided or the at least one second optical marker comprised by the image, and
for positioning of the mobile patient couch, the at least one of the drive unit or the steering unit is controlled based on the at least one of the fine position or the fine orientation.

3. The method as claimed in claim 2, wherein
for positioning the mobile patient couch in a far area, the controller controls the at least one of the drive unit or the steering unit based on the at least one of the position or the orientation, and
for positioning the mobile patient couch in a near area, the controller controls the at least one of the drive unit or the steering unit based on the at least one of the fine position or the fine orientation.

4. The method as claimed in claim 1, wherein
the controller is configured to determine a trajectory for the mobile patient couch based on at least one of the image of the at least one first optical marker, an image including at least one second optical marker, or at least one of the at least one first optical marker or the at least one second optical marker comprised by the image,
the trajectory describes a shortest, most even path of the mobile patient couch to the stationary medical device at least one of with fewest curves or that is protective of a patient, and
the controller controls the at least one of the drive unit or the steering unit based on the trajectory.

5. The method as claimed in claim 1, wherein
at least one further optical marker is arranged on at least one of walls or objects in an environment of the stationary medical device,
the optical sensor captures the at least one further optical marker in an image and provides the image including the at least one further optical marker to the controller,
the controller determines environmental information based on at least one of the image including the at least one further optical marker or the at least one further optical marker comprised by the image, and
the controller, for positioning the mobile patient couch, controls the at least one of the drive unit or the steering unit based on the environmental information.

6. The method as claimed in claim 1, wherein
at least one of the at least one first optical marker, at least one second optical marker or at least one further optical marker form an optical code,
the optical code at least one of includes or encodes marker information,
the at least one of the at least one first optical marker, the at least one second optical marker or the at least one further optical marker form a quadratic, rectangular or regular geometrical form and include a number of marking elements,
the marking elements represent simple geometrical figures and are positioned in a defined arrangement scheme, and
at least one of the marking elements or the defined arrangement scheme of the marking elements encode the marker information.

7. The method as claimed in claim 6, wherein at least one of
the regular geometrical form has corner areas, or
the marking elements are arranged in the corner areas.

8. The method as claimed in claim 6, wherein the marking elements for encoding the marker information differ at least partly in at least one of form, size or color.

9. The method as claimed in claim 6, wherein
the controller determines the marker information at least one of based on the image of at least one of the at least one first optical marker, the at least one second optical marker or the at least one further optical marker, or based on the at least one first optical marker, the at least one second optical marker or the at least one further optical marker comprised by the image, and
the controller, for positioning the mobile patient couch, at least one of controls the at least one of the drive unit or the steering unit based on the marker information, or determines a trajectory based on the marker information.

10. The method as claimed in claim 2, wherein the at least one first optical marker is larger than at least one of the at least one second optical marker or at least one further optical marker.

11. The method as claimed in claim 1, wherein the at least one first optical marker and at least one second optical marker are arranged flush with regard to a longitudinal direction of the mobile patient couch in at least one of a positioned state or a docked state.

12. A patient couch comprising:
an optical sensor; and
a controller, wherein the optical sensor and the controller are configured to carry out the method of claim 1.

13. The patient couch as claimed in claim 12, wherein the optical sensor comprises:
a camera configured to at least one of record or capture the image, wherein the camera has a capture direction aligned with a longitudinal direction of the patient couch.

14. A positioning arrangement comprising:
a movable patient couch;
a stationary medical device; and
at least one first optical marker arranged in a floor area in an environment of the stationary medical device, wherein
the movable patient couch is configured to carry out the method as claimed in claim 1.

15. The method according to claim 4, wherein the trajectory describes the shortest, most even path of the mobile patient couch to the stationary medical device for docking of the mobile patient couch at the stationary medical device.

16. The method as claimed in claim 2, wherein
the controller is configured to determine a trajectory for the mobile patient couch based on at least one of the image of the at least one first optical marker, the image including the at least one second optical marker or at least one of the at least one first optical marker or the at least one second optical marker comprised by the image,
the trajectory describes a shortest, most even path of the mobile patient couch to the stationary medical device at least one of with fewest curves or that is protective of a patient, and the controller controls the at least one of the drive unit or the steering unit based on the trajectory.

17. The method as claimed in claim 2, wherein
at least one further optical marker is arranged on at least one of walls or objects in an environment of the stationary medical device,
the optical sensor captures the at least one further optical marker in an image and provides the image including the at least one further optical marker to the controller,
the controller determines environmental information based on at least one of the image including the at least one further optical marker or the at least one further optical marker comprised by the image, and
the controller, for positioning the mobile patient couch, controls the at least one of the drive unit or the steering unit based on the environmental information.

18. The method as claimed in claim 2, wherein
at least one of the at least one first optical marker, the at least one second optical marker or at least one further optical marker form an optical code,
the optical code at least one of includes or encodes marker information,
the at least one of the at least one first optical marker, the at least one second optical marker or the at least one further optical marker form a quadratic, rectangular or regular geometrical form and include a number of marking elements,
the marking elements represent simple geometrical figures and are positioned in a defined arrangement scheme, and
at least one of the marking elements or the defined arrangement scheme of the marking elements encode the marker information.

19. The method as claimed in claim 7, wherein the marking elements for encoding the marker information differ at least partly in at least one of form, size or color.

20. The method as claimed in claim 7, wherein
the controller determines the marker information at least one of based on the image of at least one of the at least one first optical marker, the at least one second optical marker or the at least one further optical marker, or based on the at least one first optical marker, the at least one second optical marker or the at least one further optical marker comprised by the image, and
the controller, for positioning the mobile patient couch, at least one of controls the at least one of the drive unit or the steering unit based on the marker information, or determines a trajectory based on the marker information.
